(19) United States
(12) Patent Application Publication
(10) Pub. No.: US 2007/0288079 A1
(43) Pub. Date: Dec. 13, 2007

(54) Energy delivery system and uses thereof

(75) Inventors: Daniel Warren van der Weide, Madison, WI (US); Fred T. Lee JR., Madison, WI (US); Paul F. Laeseke, Madison, WI (US); Christopher L. Brace, Madison, WI (US)

Correspondence Address: MEDLEN & CARROLL, LLP, 101 HOWARD STREET, SUITE 350, SAN FRANCISCO, CA, 94105, US

(73) Assignee: Micrablate, Middleton, WI (US)

(21) Appl. No.: 11/728,428

(22) Filed: Mar. 26, 2007

Related U.S. Application Data
(60) Provisional application No. 60/785,466, filed on Mar. 24, 2006.

(51) Int. Cl. A61B 18/18

(52) U.S. Cl. 607/156; 607/115

(57) ABSTRACT

The present invention relates to systems and devices for delivering energy to tissue for a wide variety of applications, including medical procedures (e.g., tissue ablation, resection, cautery, vascular thrombosis, treatment of cardiac arrhythmias and dysrhythmias, electrosurgery, tissue harvest, etc.). In particular, the present invention relates to systems and devices for the delivery of energy with optimized characteristic impedance. In certain embodiments, methods are provided for treating a tissue region (e.g., a tumor) through application of energy with the systems and devices of the present invention.

This application claims priority to U.S. Provisional Application Ser. No. 60/785,466, filed Mar. 24, 2006, herein incorporated by reference in its entirety.

### FIELD OF THE INVENTION

The present invention relates to systems and devices for delivering energy to tissue for a wide variety of applications, including medical procedures (e.g., tissue ablation, resection, cautery, vascular thrombosis, treatment of cardiac arrhythmias and dysrhythmias, electrosurgery, tissue harvest, etc.). In particular, the present invention relates to systems and devices for the delivery of energy with optimized characteristic impedance. In certain embodiments, methods are provided for treating a tissue region (e.g., a tumor) through application of energy with the systems and devices of the present invention.

### BACKGROUND

Ablation is an important therapeutic strategy for treating certain tissues such as benign and malignant tumors, cardiac arrhythmias, cardiac dysrhythmias and tachycardia. Most approved ablation systems utilize radio frequency (RF) energy as the ablating energy source. Accordingly, a variety of RF based catheters and power supplies are currently available to physicians. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation to form on the energy emitting electrodes which limits the further deposition of electrical energy.

Microwave energy is an effective energy source for heating biological tissues and is used in such applications as, for example, cancer treatment and preheating of blood prior to infusions. Accordingly, in view of the drawbacks of the traditional ablation techniques, there has recently been a great deal of interest in using microwave energy as an ablation energy source. The advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures. Accordingly, there are a number of devices under development that utilize electromagnetic energy in the microwave frequency range as the ablation energy source (see, e.g., U.S. Pat. Nos. 4,641,649, 5,246,438, 5,405,346, 5,314,466, 5,800,494, 5,957,969, 6,471,696, 6,878,147, and 6,962,586; each of which is herein incorporated by reference in their entireties).

Unfortunately, current devices configured to deliver microwave energy have drawbacks. For example, current devices produce relatively small lesions because of practical limits in power and treatment time. Current devices have power limitations in that the power carrying capacity of the feedlines are small. Larger diameter feedlines are undesirable, however, because they are less easily inserted percutaneously and may increase procedural complication rates. In addition, heating of the feedline at high powers can lead to burns around the area of insertion for the device.

Improved systems and devices for delivering energy to a tissue region are needed. In addition, improved systems and devices capable of delivering microwave energy without corresponding microwave energy loss are needed. In addition, systems and devices capable of percutaneous delivery of microwave energy to a subject's tissue without undesired tissue burning are needed. Furthermore, systems for delivery of desired amounts of microwave energy without requiring physically large invasive components are needed.

### SUMMARY OF THE INVENTION

The present invention relates to systems and devices for delivering microwave energy to tissue for a wide variety of applications, including medical procedures (e.g., tissue ablation, resection, cautery, vascular thrombosis, intraluminal ablation of a hollow viscus, cardiac ablation for treatment of arrhythmias, electrosurgery, tissue harvest, cosmetic surgery, intraocular use, etc.). In particular, the present invention relates to systems and devices for the delivery of microwave energy with optimized characteristic impedance. In certain embodiments, methods are provided for treating a tissue region (e.g., a tumor) through application of microwave energy with the systems and devices of the present invention.

The present invention provides systems, devices, and methods that employ components for the delivery of energy at an optimized characteristic impedance. In some embodiments, the systems, devices, and methods permit delivery of desired amounts of energy with minimal power dissipation through use of an antenna having small physical dimensions to minimize invasiveness in treated tissues and organisms.

The present invention is not limited by the type of device or the uses employed. Indeed, the devices may be configured in any desired manner. Likewise, the systems and devices may be used in any application where energy is to be delivered. Such uses include any and all medical, veterinary, and research applications. However, the systems and devices of the present invention may be used in agricultural settings, manufacturing settings, mechanical settings, or any other application where energy is to be delivered.

In some embodiments, the present invention provides a device for delivery of energy, wherein the device operates with a characteristic impedance higher than 50Ω (e.g., between 50 and 90Ω; e.g., higher than 50, . . . , 55, 56, 57, 58, 59, 60, 61, 62, . . . 90Ω). In some embodiments, the characteristic impedance is 77Ω.

The device is not limited to delivering a particular type of energy. In some embodiments, the type of energy delivered by the device is microwave energy, in other embodiments the type of energy is radio frequency energy, while in other embodiments it is both.

In some embodiments, the device is configured for percutaneous, intravascular, intracardiac, laparoscopic, or surgical delivery of energy. In some embodiments, the device is configured for delivery of energy to a target tissue or region. The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the device is configured for movement and positioning, with minimal damage to the tissue or organism, at any desired location, including but not limited to, the brain, neck, chest, abdomen, and pelvis. In some embodiments, the device is configured for guided delivery, for example, by computerized tomography, ultrasound, magnetic resonance imaging, fluoroscopy, and the like.

In some embodiments, the device comprises a coaxial transmission line. The device is not limited to a particular type of coaxial transmission line. In some embodiments, the coaxial transmission line has a center conductor, a dielectric element, and an outer shield. In some embodiments, the dielectric element has near-zero conductivity. In some embodiments, the dielectric element is air, a gas, a fluid, or combination thereof. Preferably, the dielectric element lacks or substantially lacks a solid dielectric insulator. In some embodiments, the center conductor has a diameter of approximately 0.013 inches, although both larger and small diameters are contemplated. In some embodiments, the outer shield is a 20-gauge needle or a component of similar diameter to a 20-gauge needle. Preferably, the outer shield is not larger than a 16-gauge needle (e.g., no larger than an 18-gauge needle). In some embodiments, the outer shield is a 17-gauge needle. However, in some embodiments, larger devices are used, as desired. For example, in some embodiments, a 12-gauge diameter is used. The present invention is not limited by the size of the outer shield component. In some embodiments, the center conductor is configured to extend beyond the outer shield for purposes of delivering energy to a desired location. In preferred embodiments, some or all of the feedline characteristic impedance is optimized for minimum power dissipation, irrespective of the type of antenna that terminates its distal end.

The some embodiments, the systems of the present invention provide multiple feedlines and/or multiple antennas to affect one or more locations in a subject. Such application include, but are not limited to, treating large tumor masses or tumor masses having irregular shapes, where one or more of the components capable of delivered energy is inserted to a first position of a tumor and one or more of the components is inserted to a second (third, etc.) position of a tumor. In some embodiments, a first component capable of delivering energy is a first size and a second component capable of delivery energy is a second size. Such an embodiment, adds to the choices a user has in delivering the desired amount of energy for a particular application. For example, in embodiments where the size of the injury created by insertion of the device into a subject is less relevant and the tissue zone to be ablated is larger, the user may select a larger needle to deliver more energy. In contrast, where the injury associated with the insertion is to be minimized, two or more smaller needles may be used (e.g., bundled together or separately). In preferred embodiments, some or all of the feedline characteristic impedance is optimized for minimum power dissipation, irrespective of the type of antenna that terminates its distal end. In some embodiments, the device has therein multiple antenna arrays of the same or different shapes (e.g., umbrella-shaped probes, trident shaped, etc.).

In some embodiments, the system is configured to circulate a coolant (e.g., air, liquid, etc.) to help reduce undesired heating within and along the device. The present invention is not limited by the mechanism by which the cooling is applied.

In some embodiments, one or more components of the systems of the present invention may contain a coating (e.g., Teflon or any other insulator) to help reduce heating or to impart other desired properties to the component or system.

In some embodiments, the device further comprises a tuning element for adjusting the amount of energy delivered to the tissue region. In some embodiments, the tuning element is manually adjusted by a user of the system. In some embodiments, the device is pretuned to the desired tissue and is fixed throughout the procedure. In some embodiments, the tuning element is automatically adjusted and controlled by a processor of the present invention. In some embodiments, the processor adjusts the energy delivery over time to provide constant energy throughout a procedure, taking into account any number of desired factors including, but not limited to, heat, nature and/or location of target tissue, size of lesion desired, length of treatment time, proximity to sensitive organ areas, and the like. In some embodiments, the system comprises a sensor that provides feedback to the user or to a processor that monitors the function of the device continuously or at time points. The sensor may record and/or report back any number of properties, including, but not limited to, heat at one or more positions of a components of the system, heat at the tissue, property of the tissue, and the like. The sensor may be in the form of an imaging device such as CT, ultrasound, magnetic resonance imaging, or any other imaging device. In some embodiments, particularly for research application, the system records and stores the information for use in future optimization of the system generally and/or for optimization of energy delivery under particular conditions (e.g., patient type, tissue type, size and shape of target region, location of target region, etc.).

In certain embodiments, the present invention provides systems for ablation therapy, comprising a power distributor and a device for percutaneous delivery of energy to a tissue region, wherein the device operates with a characteristic impedance higher than 50Ω. In some embodiments, the power distributor includes a power splitter configured to deliver energy to multiple antennas (e.g., the same energy power to each antenna, different energy powers to different antennas). In some embodiments, the power splitter is able to receive power from one or more power distributors.

In certain embodiments, the present invention provides methods for treating a tissue region, comprising providing a target tissue or organism and a device for delivery of energy to a tissue region, wherein the device operates with a characteristic impedance higher than 50Ω. In such embodiments, the method further comprises the positioning of the device in the vicinity of the tissue region, and the percutaneous delivering of an amount of energy with the device to the tissue region. In some embodiments, the delivering of the energy results in, for example, the ablation of the tissue region and/or thrombosis of a blood vessel, and/or electroporation of a tissue region. In some embodiments, the tissue region is a tumor. In some embodiments, the tissue region comprises one or more of the heart, liver, genitalia, stomach, lung, large intestine, small intestine, brain, neck, bone, kidney, muscle, tendon, blood vessel, prostate, bladder, and spinal cord.

In some embodiments, the device is configured for percutaneous, intravascular, intracardiac, laparoscopic, or surgical delivery of energy. In some embodiments, the device is configured for delivery of energy to a target tissue or region. The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, and cauterization of blood vessels or tissue for any purposes. In some embodiments, the surgical application comprises ablation therapy (e.g., to achieve coagulation necrosis). In some embodiments, the surgical application comprises tumor ablation to target, for example, metastatic tumors. In some embodiments, the device is configured for movement and positioning, with minimal damage to the tissue or organism, at any desired location, including but not limited to, the brain, neck, chest, abdomen, and pelvis. In some embodiments, the device is configured for guided delivery, for example, by computerized tomography, ultrasound, magnetic resonance imaging, fluoroscopy, and the like.

The systems, devices, and methods of the present invention may be used in conjunction with other systems, device, and methods. For example, the systems, devices, and methods of the present invention may be used with other ablation devices, other medical devices, diagnostic methods and reagents, imaging methods and reagents, and therapeutic methods and agents. Use may be concurrent or may occur before or after another intervention. The present invention contemplates the use systems, devices, and methods of the present invention in conjunction with any other medical interventions.

### BRIEF DESCRIPTION OF THE DRAWINGS

FIG. 1 shows a schematic view of a system for microwave therapy.

FIG. 2 shows a schematic view of a device for delivering microwave energy.

FIG. 3 shows exemplary cable temperatures for various coaxial transmission lines.

### DETAILED DESCRIPTION

The present invention relates to systems and devices for delivering microwave energy to tissue for a wide variety of applications, including medical procedures (e.g., tissue ablation, treatment of arrhythmias, cautery, vascular thrombosis, electrosurgery, tissue harvest, etc.). In particular, the present invention relates to systems and devices for the delivery of microwave energy with optimized characteristic impedance. In certain embodiments, methods are provided for treating a tissue region (e.g., a tumor) through application of microwave energy with the systems and devices of the present invention.

In preferred embodiments, the systems, devices, and methods of the present invention employ microwave energy. The use of microwave energy in the ablation of tissue has numerous advantages. For example, microwaves have a broad field of power density (e.g., approximately 2 cm surrounding an antenna depending on the wavelength of the applied energy) with a correspondingly large zone of active heating, thereby allowing uniform tissue ablation both within a targeted zone and in perivascular regions (see, e.g., International Publication No. WO 2006/004585; herein incorporated by reference in its entirety). In addition, microwave energy has the ability to ablate large or multiple zones of tissue using multiple probes with more rapid tissue heating. Microwave energy has an ability to penetrate tissue to create deep lesions with less surface heating. Energy delivery times are shorter than with radiofrequency energy and probes can heat tissue sufficiently to create an even and symmetrical lesion of predictable and controllable depth. Microwave energy is generally safe when used near vessels. Also, microwaves do not rely on electrical conduction; they can radiate through tissue, fluid/blood, as well as air. Therefore, they can be used in tissue, lumins, lungs, and intravascularly.

The illustrated embodiments provided below describe the systems and devices of the present invention in terms of medical applications (e.g., ablation of tissue through delivery of microwave energy). However, it should be appreciated that the systems and devices of the present invention are not limited to medical applications. In addition, the illustrated embodiments describe the systems and devices of the present invention in terms of medical devices configured for tissue ablation. It should be appreciated that the systems and devices of the present invention are not limited to medical devices configured for tissue ablation. The illustrated embodiments describe the systems and devices of the present invention in terms of microwave energy. It should be appreciated that the systems and devices of the present invention are not limited to a particular type of energy (e.g., radiofrequency energy).

The systems and devices of the present invention provide numerous advantages over the currently available systems and devices. For example, a major drawback with currently available medical devices that utilize microwave energy is the undesired dissipation of the energy through transmission lines onto a subject's tissue resulting in undesired burning. Such microwave energy loss results from limitations within the design of currently available medical devices. In particular, medical devices utilizing microwave energy transmit energy through coaxial cables having therein a dielectric material (e.g., polyfluorothetraethylene or PTFE) surrounding an inner conductor. Dielectric materials such as PTFE have a finite conductivity, which result in the undesired heating of transmission lines. This is particularly true when one supplies the necessary amounts of energy for a sufficient period of time to enable tissue ablation. The present invention provides systems, devices, and method that overcome this limitation. In particular, the present invention provides devices lacking, or substantially lacking, a solid dielectric insulator. For example, using air in place of a traditional dielectric insulator results in an efficient device operating at 77Ω. In some embodiments, the devices employ a near-zero conductivity dielectric material (e.g., air, water, inert gases, vacuum, partial vacuum, or combinations thereof). The present invention is not limited by the means by which the higher impedance devices are generated. As described in more detail below, the overall temperature of the transmission lines within the medical devices of the present invention are greatly reduced through use of coaxial transmission lines with near-zero conductivity dielectric materials, and therefore, greatly reduces undesired tissue heating.

Thus, in some embodiments, the systems and devices of the present invention are provided with a high characteristic impedance (e.g., between 50 and 90Ω; e.g., higher than 50, . . . , 55, 56, 57, 58, 59, 60, 61, 62, . . . 90Ω, etc.). Standard impedance for coaxial transmission lines within medical devices is 50Ω or lower. Generally, coaxial transmission lines with impedance lower than 50Ω have high amounts of heat loss due to the presence of dielectric materials with finite conductivity values. As such, medical devices with coaxial transmission lines with impedance at 50Ω or lower have high amounts of heat loss along the transmission lines. The present invention overcomes this problem by utilizing a coaxial transmission line with a dielectric material having near-zero conductivity (e.g., air) and other methods for achieving the same end.

In addition, by providing a coaxial transmission line with a dielectric material having near-zero conductivity, and avoiding the use of typical dielectric polymers, the coaxial transmission line may be designed such that it can fit within small needles (e.g., 18-20 gauge needles). Typically, medical devices configured to delivery microwave energy are designed to fit within large needles due to bulky dielectric materials. Microwave ablation has not been extensively applied clinically due to the large probe size (14 gauge) and relatively small zone of necrosis (1.6 cm in diameter) (Seki T et al., Cancer 74:817 (1994)) that is created by the only commercial device (Microtaze, Nippon Shoji, Osaka, Japan. 2.450 MHz, 1.6 mm diameter probe, 70 W for 60 seconds). Other devices use a cooling external water jacket that also increases probe size and can increase tissue damage. These large probe sizes increase the risk of complications when used in the chest and abdomen. In some embodiments of the present invention, the maximum outer diameter of the portion of the device that enters a subject is 16-18 gauge or less.

Systems and devices employing a characteristic impedance of greater than 50Ω (e.g., approximately 77Ω) of the present invention finds use in any type of medical devices where over heating of transmission lines is to be reduced or avoided.

Certain preferred embodiments of the present invention are described below. The present invention is not limited to these embodiments.

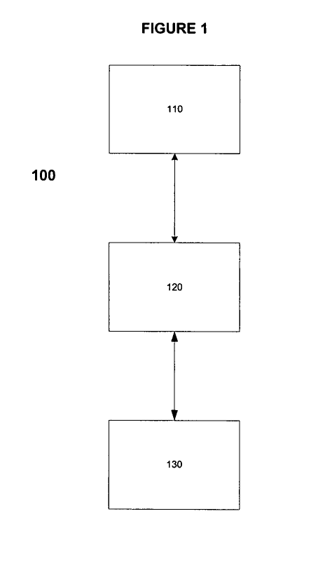

FIG. 1 shows a schematic view of a system for microwave therapy 100 that operates with a characteristic impedance of approximately 77Ω (e.g., between 50 and 90Ω; e.g., higher than 50, . . . , 55, 56, 57, 58, 59, 60, 61, 62, . . . 90Ω, etc.). The system for microwave therapy 100 is not limited to a particular type of microwave therapy. Indeed, the system for microwave therapy 100 encompasses any type of microwave therapy (e.g., exposure of a tissue (e.g., cancer cells) to high temperatures so as to kill the tissue or to make the tissue more sensitive to alternative treatment forms (e.g., to render tissue more sensitive to the effects of radiation; to render tissue more sensitive to anticancer drugs). In some embodiments, the system for microwave therapy 100 generally comprises a generator 110, a power distribution system 120, and an applicator device 130.

Still referring to FIG. 1, in some embodiments, the generator 110 serves as an energy source to the system for microwave therapy 100. In some embodiments, the generator 110 is configured to provide as much as 100 watts of microwave power of a frequency of 2.45 GHz, although the present invention is not so limited. The system for microwave therapy 100 is not limited to a particular type of generator 110. Exemplary generators that find use with the present invention include, but are not limited to, those available from Cober-Muegge, LLC, Norwalk, Conn., USA.

Still referring to FIG. 1, in some embodiments, the generator 110 has therein a power output port operating at a characteristic impedance of approximately 77Ω (e.g., between 50 and 90Ω; e.g., higher than 50, . . . , 55, 56, 57, 58, 59, 60, 61, 62, . . . 90Ω, etc.). In some embodiments, the components within the generator 110 have a characteristic impedance of approximately 77Ω or may be transformed to a characteristic impedance of approximately 77Ω. In some embodiments, the generator 110 has therein a magnetron source with a characteristic impedance of 77Ω, which drives a directional coupler and coaxial connector (output port) that are all at 77Ω. In some embodiments, the generator 110 has therein a magnetron source with a characteristic impedance of approximately 50Ω (e.g., 45Ω, 47Ω, 49Ω, 51Ω, 53Ω) but may be transformed to the approximately 77Ω using, for example, transmission line transformers.

Still referring to FIG. 1, in some embodiments, the power distribution system 120 distributes energy from the generator 110 to the applicator device 130. The power distribution system 120 is not limited to a particular manner of collecting energy from the generator 110. The power distribution system 120 is not limited to a particular manner of providing energy to the applicator device 130. In some embodiments, the power distribution system 120 operates at an impedance of approximately 77Ω. In some embodiments, the power distribution system 120 is configured to transform the characteristic impedance of the generator 110 such that it matches the characteristic impedance of the applicator device 130 (e.g., 77Ω).

Still referring to FIG. 1, in some embodiments, the applicator device 130 is configured to receive microwave energy from the power distribution system 120 and deliver the microwave energy to a load (e.g., tissue). In some embodiments, the applicator device 130 operates at a characteristic impedance of 77Ω. In some embodiments, the applicator device 130 is configured to transform the characteristic impedance of power distribution system 120 such that it matches the characteristic impedance level of the applicator device 130 (e.g., 77Ω).

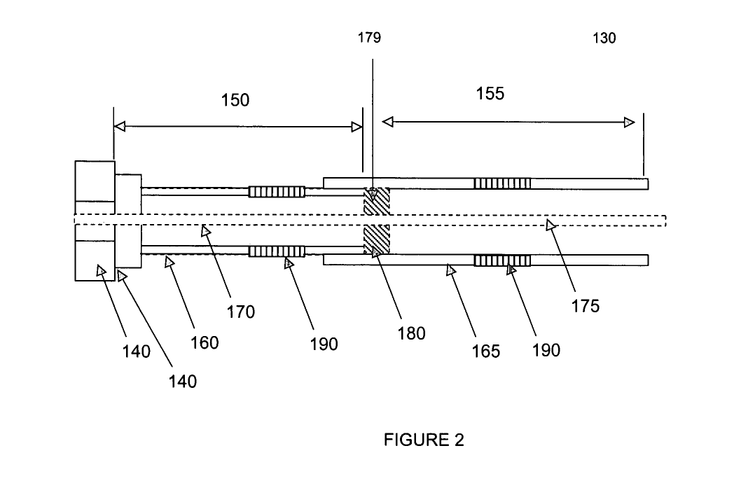

FIG. 2 shows a schematic drawing of an applicator device 130. One skilled in the art will appreciate any number of alternative configurations that accomplish the physical and/or functional aspects of the present invention. As shown in FIG. 2, the applicator device 130 comprises a proximal coaxial transmission line 150 and a distal coaxial transmission line 155.

Still referring to FIG. 2, the proximal coaxial transmission line 150 and the distal coaxial transmission line 155 are not limited to a particular type of material. In some embodiments, the proximal coaxial transmission line 150 and the distal coaxial transmission line 155 are constructed from commercial-standard 0.047-inch semi-rigid coaxial cable whose polymer dielectric has been removed. In some embodiments, the proximal coaxial transmission line 150 and the distal coaxial transmission line 155 are silver-plated, although the present invention is not so limited. The proximal coaxial transmission line 150 and the distal coaxial transmission line 155 are not limited to a particular length.

Still referring to FIG. 2, in some embodiments, the proximal coaxial transmission line 150 has a proximal coaxial outer shield 160. In some embodiments, the proximal coaxial transmission line 150 has a proximal coaxial center conductor 170. In some embodiments, the proximal coaxial center conductor 170 is configured to conduct cooling fluid along its length. In some embodiments, the proximal coaxial center conductor 170 is hollow. In some embodiments, the proximal coaxial center conductor 170 has a diameter of, for example, 0.012 inches. In some embodiments, the proximal coaxial transmission line 150 is lacking a polymer dielectric layer. In some embodiments, the proximal coaxial transmission line 150 utilizes a dielectric material with near-zero conductivity (e.g., air, gas, fluid). In some embodiments, the proximal coaxial transmission line 150 has a characteristic impedance of approximately 64.2Ω or more. Experiments conducted during the development of the present invention demonstrated that a proximal coaxial center conductor 170 with a dielectric material of near-zero conductivity (e.g., air) and a diameter of approximately 0.012 inches results in increased impedance (e.g., 64.2Ω) for the proximal coaxial transmission line 150. Increased impedance for the proximal coaxial transmission line 150 permits use of the applicator device 130 without undesired heating along the proximal coaxial transmission line 150.

Still referring to FIG. 2, in some embodiments, the distal coaxial transmission line 155 has a distal coaxial outer shield 165. In some embodiments, the distal coaxial transmission line 155 has a distal coaxial center conductor 175. In some embodiments, the distal coaxial center conductor 175 is configured to conduct cooling fluid along its length. In some embodiments, the distal coaxial center conductor 175 is hollow. In some embodiments, the distal coaxial center conductor 175 has a diameter of, for example, 0.013 inches. In some embodiments, the distal coaxial transmission line 155 is lacking a polymer dielectric layer. In some embodiments, the distal coaxial transmission line 155 utilizes a dielectric material with near-zero conductivity (e.g., air, gas, fluid). In some embodiments, the distal coaxial transmission line 155 has a characteristic impedance of approximately 77Ω. Having a distal coaxial center conductor 175 with a dielectric material of near-zero conductivity (e.g., air) and a diameter of approximately 0.013 inches results in increased impedance (e.g., 77Ω) for the distal coaxial transmission line 155. Increased impedance for the distal coaxial transmission line 155 permits use of the applicator device 130 without undesired heating along the distal coaxial transmission line 155.

Still referring to FIG. 2, the distal coaxial transmission line 155 is configured to mate with the proximal coaxial transmission line 150. In some embodiments, the proximal coaxial transmission line 150 fits within the distal coaxial transmission line 155 such that the outer distal coaxial outer shield 165 is positioned on the outside of the proximal coaxial outer shield 160. In some embodiments, the proximal coaxial center conductor 170 is aligned with the distal coaxial center conductor 175. In some embodiments, the proximal coaxial center conductor 170 is aligned with the distal coaxial center conductor 175 with a dielectric bead 180. The applicator tool 130 is not limited to a particular type or size of dielectric bead 180 (e.g., epoxy bead, ceramic bead, Teflon bead, delrin bead).

Still referring to FIG. 2, the distal coaxial outer shield 165 is not limited to a particular function. In some embodiments, the distal coaxial outer shield 165 serves as a needle for insertion into a subject. The distal coaxial outer shield 165 is not limited to a particular material composition. In some embodiments, the material composition of the distal coaxial outer shield 165 is stainless steel. In some embodiments, the material composition of the distal coaxial outer shield 165 is silver plated stainless steel. The distal coaxial outer shield 165 is not limited to a particular size. In some embodiments, the size of the distal coaxial outer shield 165 is of a 17 gauge needle or smaller. In some embodiments, the size of the distal coaxial outer shield 165 is of a 20 gauge needle or smaller.

Still referring to FIG. 2, in some embodiments, the overlap between the proximal coaxial transmission line 160 and the distal coaxial transmission line 165 serves as a slidable joint 179. In some embodiments, the slidable joint 179 allows for telescoping (e.g., extending) the distal coaxial center conductor 175 beyond the distal end of the distal coaxial outer shield 165. Upon such extension, the distal coaxial center conductor 165 serves as a resonant monopole antenna wherein the electric field peaks at the end of the exposed distal coaxial center conductor 165. The distal coaxial center conductor 165 is not limited to a particular amount of extension. In some embodiments, the distal coaxial center conductor 165 is exposed to a length so as to assure that impedance matching with the transmission lines. In use, the exposed distal coaxial center conductor 165 is applied to a subject's tissue for purposes of treatment (described in more detail below). The slidable joint 179 further permits the tuning of the applicator device 130 such that the impedance level between the proximal coaxial transmission line 150 and the distal coaxial transmission line 155 may be adjusted.

Still referring to FIG. 2, the proximal coaxial outer shield 160 and the distal coaxial outer shield 165 have therein breather sections 190 (e.g., mesh or slotted breather sections). The breather sections 190 are not limited to a particular type or size. In some embodiments, the breather sections 190 serve to allow the exhaust of, for example, a cooling fluid or gas.

The systems and devices of the present invention may be combined within various system/kit embodiments. For example, the present invention provides kits comprising one or more of a generator, a power distribution system, and an applicator device, along with any one or more accessory agents (e.g., surgical instruments, software for assisting in procedure, processors, temperature monitoring devices, etc.). The present invention is not limited to any particular accessory agent. Additionally, the present invention contemplates kits comprising instructions (e.g., ablation instructions, pharmaceutical instructions) along with the systems and devices of the present invention and/or a pharmaceutical agent (e.g., a sedating medication, a topical antiseptic, a topical anesthesia).

The devices of the present invention may be used in any medical procedure (e.g., percutaneous or surgical) involving delivery of energy (e.g., microwave energy) to a tissue region. The present invention is not limited to a particular type or kind of tissue region (e.g., brain, liver, heart, blood vessels, foot, lung, bone, etc.). For example, the systems of the present invention find use in ablating tumor regions. In such uses, the applicator device is inserted into, for example, a subject such that the distal end of the distal coaxial outer shield is positioned in the vicinity of the desired tissue region. Next, the generator is used to provide a desired amount of microwave energy to the power distribution system at a characteristic impedance level, which in turn provides the energy at a characteristic impedance level to the applicator device. Next, through use of a visualizing agent, the distal coaxial center conductor is extended from the distal coaxial outer shield in a manner retaining the characteristic impedance level. Next, a desired amount of microwave energy is delivered to the desired tissue region (e.g., tumor) generating an electric field of sufficient strength to ablate the desired tissue region. Due to the characteristic impedance level maintained throughout the transmission lines of the applicator device, the overall temperature of the transmission lines is greatly reduced, resulting in a reduced chance for undesired tissue overheating. The present invention further provides methods involving the simultaneous use of multiple (e.g., two or more) applicator devices for the treatment of a tissue. The present invention further provides methods involving the simultaneous use of multiple (e.g., two or more) applicator devices for the treatment of a tissue. In some embodiments, the present invention provides methods wherein the simultaneous use of multiple antennas are phased to achieve constructive and destructive interference (e.g., for purposes of selectively destroying and sparing portions of a tissue region).

In some embodiments, the present invention further provides software for regulating the amount of microwave energy provided to a tissue region through monitoring of the temperature of the tissue region (e.g., through a feedback system). In such embodiments, the software is configured to interact with the systems for microwave therapy of the present invention such that it is able to raise or lower (e.g., tune) the amount of energy delivered to a tissue region. In some embodiments, the type of tissue being treated (e.g., liver) is inputted into the software for purposes of allowing the software to regulate (e.g., tune) the delivery of microwave energy to the tissue region based upon pre-calibrated methods for that particular type of tissue region. In other embodiments, the software provides a chart or diagram based upon a particular type of tissue region displaying characteristics useful to a user of the system. In some embodiments, the software provides energy delivering algorithms for purposes of, for example, slowly ramping power to avoid tissue cracking due to rapid out-gassing created by high temperatures. In some embodiments, the software allows a user to choose power, duration of treatment, different treatment algorithms for different tissue types, simultaneous application of power to the antennas in multiple antenna mode, switched power delivery between antennas, coherent and incoherent phasing, etc.

In some embodiments, the software is configured for imaging equipment (e.g., CT, MRI, ultrasound). In some embodiments, the imaging equipment software allows a user to make predictions based upon known thermodynamic and electrical properties of tissue and location of the antenna(s). In some embodiments, the imaging software allows the generation of a three-dimensional map of the location of a tissue region (e.g., tumor, arrhythmia), location of the antenna(s), and to generate a predicted map of the ablation zone.

### EXAMPLES

### Example I

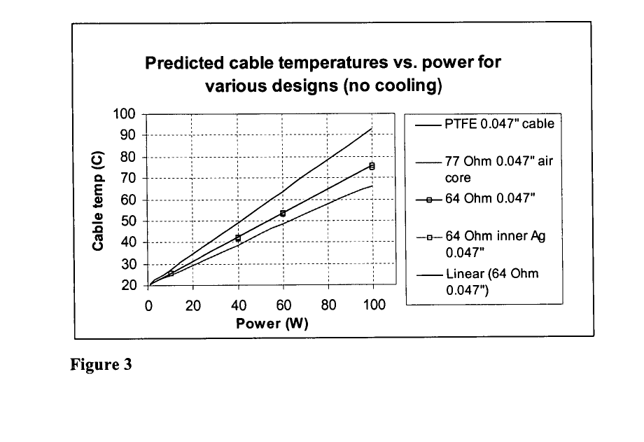

The power loss of several coaxial transmission lines with different combinations of polyfluorotetraethylene (PTFE) dielectric material, air dielectric material, copper conductors and silver conductors was examined. As shown in FIG. 3, a standard copper conductor with a PTFE dielectric cable yielded the highest temperature (˜92° C. at 100 W input power). Removing the PTFE dielectric gave an impedance of 64Ω, which resulted in a lower temperature (˜76 C at 100 W) that was unchanged whether copper (Cu) or silver (Ag) was used for the inner conductor. The lowest temperature (˜66° C. at 100 W) resulted from changing the inner-to-outer conductor diameter ratio to create a 77 Ωohm cable with air dielectric.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A device comprising an antenna configured for delivery of energy to a tissue, wherein said device operates with a characteristic impedance higher than 50Ω.

2. The device of claim 1, wherein said energy is microwave energy.

3. The device of claim 1, wherein said characteristic impedance is between 50 and 90Ω.

4. The device of claim 1, wherein said characteristic impedance is 77Ω.

5. The device of claim 1, wherein said device comprises a coaxial transmission line.

6. The device of claim 5, wherein said coaxial transmission line has a center conductor, a dielectric element, and an outer shield.

7. The device of claim 6, wherein said dielectric element has near-zero conductivity.

8. The device of claim 6, wherein said dielectric element is selected from the group consisting of air, gas, and fluid.

9. The device of claim 6, wherein said center conductor has a diameter of approximately 0.013 inches or less.

10. The device of claim 6, wherein said outer shield has a diameter equal to or less than a 20-gauge needle.

11. The device of claim 1, further comprising a tuning element for adjusting the amount of energy delivered to said tissue region.

12. The device of claim 1, wherein said device is configured to deliver a sufficient amount of energy to ablate said tissue region or cause thrombosis.

13. A system for ablation therapy, comprising a power distributor and a device for delivery of energy to a tissue region, wherein said device operates with a characteristic impedance higher than 50Ω.

14. The system of claim 13, wherein said energy is microwave energy.

15. The system of claim 13, wherein said characteristic impedance is between 50 and 90Ω.

16. The system of claim 13, wherein said characteristic impedance is 77Ω.

17. The system of claim 13, wherein said device comprises a coaxial transmission line.

18. The system of claim 17, wherein said coaxial transmission line has a center conductor, a dielectric element, and an outer shield.

19. The system of claim 17, wherein said dielectric element has near-zero conductivity.

20. The system of claim 28, wherein said dielectric element is selected from the group consisting of air, liquid and gas.

21. The system of claim 13, further comprising a generator operating at a characteristic impedance of between 50 and 90Ω.

22. The system of claim 13, wherein said power distributor has characteristic impedance between 50 and 90Ω.

23. A method of treating a tissue region, comprising:
a) providing a tissue region and a device for delivery of energy to a tissue region, wherein said device operates with a characteristic impedance higher than 50Ω;
b) positioning said device in the vicinity of said tissue region,
c) delivering an amount of energy with said device to said tissue region.

24. The method of claim 23, wherein said tissue region is a tumor.

25. The method of claim 23, wherein said energy is microwave energy.

26. The method of claim 23, wherein said characteristic impedance is between 50 and 90Ω.

27. The method of claim 23, wherein said characteristic impedance is 77Ω.

28. The method of claim 23, wherein said device comprises a coaxial transmission line.

29. The method of claim 28, wherein said coaxial transmission line has a center conductor, a dielectric element, and an outer shield.

30. The method of claim 29, wherein said dielectric element has near-zero conductivity.

31. The method of claim 29, wherein said dielectric element is selected from the group consisting of air, gas and liquid.
